(19) United States
(12) Patent Application Publication
(10) Pub. No.: US 2022/0215542 A1
(43) Pub. Date: Jul. 7, 2022

(54) METHOD, DEVICE AND COMPUTER-READABLE MEDIUM FOR AUTOMATICALLY DETECTING HEMODYNAMICALLY SIGNIFICANT CORONARY STENOSIS

(71) Applicant: Jean-François PAUL, BOURG-LA-REINE (FR)

(72) Inventor: Jean-François PAUL, BOURG-LA-REINE (FR)

(73) Assignee: AXE PI, Nice (FR)

(21) Appl. No.: 17/613,458

(22) PCT Filed: May 18, 2020
(86) PCT No.: PCT/EP2020/063797

§ 371 Date: Nov. 22, 2021

(30) Foreign Application Priority Data
May 23, 2019 (FR) FR1905415

(51) Int. Cl. G06T 7/00; G06T 7/11; G06V 10/82; G06V 10/25; G06N 3/04; G16H 30/20; G16H 50/20; G16H 50/30

(52) U.S. Cl. CPC G06T 7/0012; G06T 7/11; G06V 10/82; G06V 10/25; G06N 3/0454; G06T 2207/20084; G16H 50/20; G16H 50/30; G06T 2207/10081; G06T 2207/30048; G16H 30/20

(57) ABSTRACT

A computer-implemented method for determining the presence of a coronary stenosis for a patient, including a step of receiving at least one curvilinear or stretched multiplanar medical CT (X-scanner) image of the patient including the coronary stenosis as well as a step of detecting the coronary stenosis on the image or on a part of the image by using a first deep neural network. The method further includes a step of predicting a coronary reserve flow value interval (FFR for fractional flow reserve) by using a second trained deep neural network, applied directly to the images or parts of images detected and/or by manual, semi-automated and/or automated measurement of at least two morphological criteria selected from the minimum diameter of the stenosis, the minimum surface area of the stenosis, the degree of maximum coronary stenosis in diameter, the degree of maximum coronary stenosis in surface area, the length of the stenosis and/or the myocardial mass and the percentage of myocardial mass downstream from the coronary stenosis.

The present invention relates to a computer-implemented method for automatically determining the presence of a hemodynamically significant coronary stenosis by predicting the value of FFR (fractal flow reserve) associated with the stenosis thus detected, as well as a device capable of automatically determining the presence of a hemodynamically significant coronary stenosis by predicting the value of FFR (fractional flow reserve) associated with the stenosis thus detected and a non-transitory computer-readable medium storing computer-readable program instructions for automatically determining the presence of a hemodynamically significant coronary stenosis by predicting the value of FFR associated with the stenosis thus detected.

Coronary disease is the second cause of mortality in the developed countries, after cancer. In particular, it affects more than fifteen millions of American. It can be revealed in a noisy manner, that is far the first cause of sudden death in the world. About 50,000 case of sudden death per year in France, most by myocardial infarction. It can reach young subjects, sometimes in their thirties. Its incidence increases with the aging of the population and the development of chronic diseases such as diabetes or arterial hypertension.

Cardiovascular diseases (CVD) regroup a number of disorders affecting the heart and blood vessels as:
arterial hypertension (voltage rise);
coronary heart disease (heart attack or infarction);
cerebrovascular diseases (stroke);
peripheral arteriopathies;
heart failure;
rheumatic heart diseases;
congenital heart disease;
cardiomyopathy.

Coronary cardiopathies also called coronary heart diseases, or coronary insufficiency, are obstructive diseases of the coronary arteries which vascularize the heart.

When it moves to stenosis (narrowing to occlusion), coronary artery injury results in coronary artery disease, or coronary artery disease, or coronary heart failure.

Coronary deficiencies generally result in myocardial ischemia, i.e., insufficient blood supply (ischemia) to the cardiac muscle (myocardial), in particular due to vascular obstruction.

Numerous complementary examinations allow to explore the myocardial ischemia, the main examinations being the electrocardiogram, the stress test, the MRI, the myocardial scintigraphy, and the coronarography, and more recently the coronary angiography.

In practice, the classification of coronary stenoses according to their severity is most often visually performed in coronary angiography: it depends on a segmentation which relies on the extraction of the central line of the vessel. It is dependent on the experience of the reader. Stenosis is considered usually significant for a reduction in diameter of at least 50%, by visual estimation. This visual evaluation remains inaccurate with substantial inter-observer variability.

The accuracy in the classification of the degree of severity is very related to the image quality. The image quality depends on the heart rate, on potential staircase artifacts between two beats, on the quality of the contrast agent injection, on the noise level in the image, and on the possible presence of severe calcifications. The last cardiac scanner technology allows to obtain the best image quality by reducing most of the artefacts cited.

A long reading experience is necessary for the analysis of a Coronary angio-scanner (Kerl et al, “64-Slice Multidetector-Row Computer Tomograph in the Diagnosis of Coronary Artery Disease: Interview Agreement Among Radiologist With Varied Level of experience on Per-patient and Per-segment Basis.” J Thorac Imaging January 2012; 27 (1): 29-35).

These situations can lead to wrongly concluding to coronary stenosis, sometimes leading to unnecessarily performing a conventional, invasive and expensive angiography.

Coronary angiography (or CCTA for Coronary CT angiography) is a more recent, highly sensitive method for the non-invasive detection of patients who are suspected of having a coronary artery disease, with a very high negative predictive value (typically greater than 95%). Being the most sensitive method, it tends to be used in the screening of coronary diseases as first intention examination.

On the other hand, the coronary angiograph has a least good specificity (about 50-70%) due to frequent false positives. Its positive predictive value of the scanner is therefore lower. The cases of false positives are observed in particular in the case of coronary calcifications and/or in the event of motion artifacts during the acquisition of the images. The latter may increase or even create stenoses (inexistent) on the image. Thus, reading expertise is required to minimize the number of false positives. Good expertise is acquired in several years (5 years at minimum) for radiologists or cardiologists working in a dedicated center in cardiac imaging.

Document The SCOT-HEART Investors, “Coronary CT Angiography and 5-Yeaar Rik of Myocardial Infarct” N Engl J Med. 6 Sep. 2018, described in particular that the use of coronary angiograph can reduce the rate of infarct and mortality compared to a standard evaluation by a conventional force test.

However, the growing use of the coronary angiograph is, in practice, braked by the level of expertise required for reliable interpretation in the current practice.

The arrival of artificial Intelligence (AI) allows to envisage transferring certain elements of the medical expertise in algorithmic form. Machine Learning tools and in particular the neural networks (NN) allow to reproduce expertise, which is very useful in the field of image recognition. Therefore, multiple projects are developed in the field of medical imaging.

Thus, the present invention includes adapting expertise in the reading of coronary angiograph using the AI techniques.

Publication Zreik M et al, “A Recurrent CNN for Automated detection and Classification of Coronary Artery Plate and Stenosis in Coronary CT Angiography.” I EEE Trans Med Imaging 2018 discloses a first method for automatic detection of stenoses by machine learning. However, this method uses only stretched MPR images (multiplanar reconstructions), and analyzes the arteries by volume fragments, with a three-level classification (normal, less than 50% and greater than 50%) without automated evaluation of the image quality, and does not associate any functional evaluation.

The application US20150112182 discloses the prediction of FFR by using Artificial Intelligence (by machine learning) from any medical image type, generally. In this document, the use of the scanner imaging is mentioned in examples just like the ultrasound or the MRI. On the other hand, this document does not mention the type of scanner image chosen for learning, the aggregated analysis of multiple images of the same lesion under several incidences, the number of images for the analysis, and/or the influence of the image quality.

According to the document Tonino et al, “Fractal Flow Reserve versus Angiography for Guiding Percutaneous Coronary Intervention.” N Engl J Med. 15 Jan. 2009 measuring PPR during coronary angiography is a reference examination to determine whether coronary stenosis is functionally significant, with a threshold at 0.8. Indeed, it has been shown in patients with stable angina and which had a FFR of less than or equal to 0.8, that coronary angioplasty significantly reduces the subsequent need for urgent revascularization. In the contrary case, the angioplasty does not have a benefit in terms of prognosis. In another study, the frequency of major cardiac events after angioplasty was reduced by 28% if the intervention was decided according to the value of FFR (less than or equal to 0.8) relative to the single visual interpretation of the coronary angiography images.

Coronary angiograph (CCTA) is a sensitive method for the detection of coronary stenoses, making it convenient to spread out coronary stenosis when the examination is normal. On the other hand, the coronary angiograph tends to overestimate the degree of coronary stenosis, leading to a less good specificity of this tool. The difference between the visual degree of a stenosis and its hemodynamic effect (therefore its functional character) is known and confirmed in recent studies comparing different estimates of stenoses to their FFR value: in the FAME test, the FFR was greater than 0.8 per 63% of the intermediate estimated stenoses (50-70%), and for 20% of the cases for the severe estimated stenoses visually (between 70 and 99%) (Tonino et al. “Angiography Versus Function of Coronary Artery Stenoses in the FAME Study.” J Am Coll Cardiol. June 2010).

Thus, a non-invasive method that would provide reliable both anatomical and functional information would reduce the need for a conventional invasive angiography with any invasive measurement of FFR, would be desirable.

The present invention relates to an FFR prediction based on Artificial Intelligence Analysis applied to coronary stenosis images observed in a scanner under multiple incidences, associated or not with a combination of at least two anatomical criteria.

Other approaches use machine learning techniques for FFR prediction using a wide database of coronary anatomies generated from a model. This type of approach is, in particular, disclosed in the document Tesla et al, “Coronary CT angiograph derived morphologic and quantitative functional plate markers correlated with invasive fractal flow reserve for detecting hemodynamically mean stenosis.” J Cardiovasc Comput Tomogr. However, in this document the used coronary images do not originate from true patients. Their prediction is based on the principle of fluid hemodynamics.

Moreover, the document Nakanishi et al, “Automated estimation of quality image for coronary computed tomography using machine learning.” Eur Radiol. September 2018 describes the use of deep learning for the automatic evaluation of the image quality in CCTA (coronary CT angiography). However, in this document, poor quality examinations were few due to an artificial preselection that does not correspond to daily practice. Moreover, in this document, only axial, coronal and sagittal images were analyzed, but no MPR images. However, the MPR images which are the basis of the X-ray interpretation in current practice.

Document Lossau et al, “Motion artifact recognition and quantification in coronary CT angiograph using convolutional neural convolution.” Med Image Anal. February 2019, also describes the use of deep learning for the automatic evaluation of the image quality in CCTA. This document discloses the feasibility of deep learning to quantify cardiac motion artifacts. On the other hand, in this document, the overall quality of the image is not analyzed (including noise, low contrast, or large calcifications). It is the overall quality of the image which allows to provide a diagnostic confidence index taking into account all the parameters interfering with the image.

The present invention relates to a computer-implemented method for automatically determining the presence of a hemodynamically significant coronary stenosis by predicting an interval of the FFR value associated with the stenosis thus detected, and a device capable of automatically determining the presence of a hemodynamically significant coronary stenosis by predicting an interval of the FFR value associated with the stenosis thus detected, and a non-transitory computer-readable medium storing computer-readable program instructions for automatically determining the presence of a hemodynamically significant coronary stenosis by predicting an interval of the FFR value associated with the stenosis thus detected.

The studies published in CCTA rely on visual estimates of the stenoses, at the threshold of 50% in diameter. The relevance of this detection is very dependent on the observer and its level of reading experience.

At this time, there is no reliable tool for automatic detection of coronary lesions at the threshold of 50% available in current practice, due to the multiple factors interfering with the interpretation. These multiple factors are in particular the following: contrast, noise, cardiac or respiratory motion, anatomical variation, calcifications, which make the interpretation difficult.

The artificial intelligence techniques use statistical models capable of reproducing expertise, long to be acquired. By driving a neural network on thousands of images labeled by an expert, it is possible to approach the level of this expert without a priori modeling. Thus, it is possible to provide automatic detection of coronary stenosis with a performance close to that of an expert.

It is what the applicant was able to validate. The ability to detect stenoses thus exceeds 90% in the validation data.

It is from this threshold of about 50% in diameter that a lesion risks limiting the coronary flow. The stenosis is called significant in this case. One speaks then of a hemodynamically significant coronary stenosis.

Beyond the detection of the stenosis at the threshold, it is very important to know whether the stenosis is hemodynamic or not. Indeed, only the stenoses which cause a pressure drop downstream of the stenosis must be treated mechanically by stent or possibly by coronary bypass.

This hemodynamic effect can be measured by measuring the pressure drop in maximum hyperemia. It is the basis of the calculation of the FFR.

Studies have shown that there is a benefit to mechanically treat patients with coronary stenosis only if the FFR is less than or equal to 0.8 translating a pressure drop downstream of the stenosis.

Therefore, it is important to be able to predict a level of FFR less than or equal to 0.8 in front of a coronary stenosis image. Such prediction allows to avoid other more or less invasive examinations, expensive and the performance of which are not always correlated with the results of the invasive FFR.

If the FFR begins to drop for stenoses from 40-50%, it is recognized that the degree of stenosis on the image does not allow to correctly and reliably predict the value of the FFR.

Between 50 and 70% of stenoses, two thirds of the patients have FFRs greater than 0.8, therefore their lesions are not hemodynamically significant. Between 70 and 99% of stenoses, 80% of the patients have, on the other hand, an FFR of less than or equal to 0.8, the stenosis is therefore hemodynamically significant.

Thus, in the event of intermediate stenosis, that is to say comprised between 50 and 80%, it is difficult to know whether to treat or not the patient by a mechanical system (stent), or by bridging. Indeed, on this day, it is not possible to determine the hemodynamic character or not of a stenosis on the single anatomical imaging.

Other anatomical criteria have been proposed in the literature but do not appear to be sufficiently reliable to predict a hemodynamic effect of a stenosis.

These anatomical criteria include, in particular, the degree of stenosis on the surface or in diameter, the minimum diameter, the minimum luminal surface, or the length of the stenosis. However, these anatomical criteria do not appear to have been studied in association but only in an isolated manner.

The applicant has been able to demonstrate that certain combinations of anatomical criteria were very relevant to determine the value of the FFR above or below the threshold of 0.8. This determination allows, in particular, to opt or not for a stent or bridge treatment.

Moreover, the direct learning of a neural network of the images associated with FFR values also allows such prediction on new images. The anatomical predictions and by Artificial Intelligence appear complementary, and their association appears to be very efficient.

The imaging of stress (ultrasound, by MRI or by scanner) is used to highlight the hemodynamic character of a stenosis of other techniques based on coronary angiograph alone, without using stress imaging, have also been proposed:
the use of the anatomical features of the stenosis, without obtaining evidence results, since the criteria have been studied separately and not in association as in the present invention;
the attenuation gradient beyond the stenosis (TAG for transluminal attenuation gradient), which is not used in practice;
an estimate of the FFR from a Computational Fluid Dynamics (CFD). This last model is known as FFR-CT (for Fractal flow reserve computer tomography). The simulation techniques require important computing resources, the results of PPR-CT request 24 h processing. Moreover, the results depend on the image quality, up to 20% of the cases have been excluded from studies on the FFR-CT due to insufficient image quality.

Other methods exist but also have disadvantages. It is, for example, methods using pharmacological stress (Adenosine, Dipyridamole, or Dobutamine), such as stress MRI, which is expensive, time consuming and inexpensive. Moreover, the injection of Gadolinium could expose long-term risks according to recent publications.

The stress scanner is the most recent application, without large-scale evaluation at the current time. It induces an additional cost, an injection of iodine and an irradiation which can be important due to repetitive acquisitions necessary for obtaining an infusion curve.

The nuclear medicine imaging also involves an irradiation dose to the patient linked to the injected radiotracer.

The stress ultrasonography requires a driven operator, and its sensitivity appears to be low.

Considering the above, a problem addressed by the present invention is in particular to automatically detect potentially significant stenosis with good reliability and then to predict the PPR directly on anatomical images, when the stenosis is visually potentially significant, without the need for complex modeling or addition of other imaging examination. This has, in particular, the advantage of being able to achieve a considerable saving in the level of health systems everywhere in the world by greatly simplifying the patient care path.

The solution to this problem addressed is a computer-implemented method for determining the presence of coronary stenosis for a patient, comprising:
a step of receiving at least one curved or stretched multiplanar medical image of computed tomography (X-scanner) of said patient including coronary stenosis;
a step of detecting said coronary stenosis on said image or on a portion of said image by using a first trained deep neural network;
   characterized in that it further comprises:
a step of predicting a coronary fractal flow reserve value interval (FFR) by manual, semi-automated and/or automated measurement of at least two morphological criteria chosen from:
   the minimum diameter of the stenosis in mm;
   the minimum surface area of the stenosis in mm²;
   the degree of maximum coronary stenosis expressed as percentage (%) of diameter;
   the degree of maximum coronary stenosis expressed in percentage (%) of surface;
   the length of the stenosis in mm; and/or
   the myocardial mass or the percentage (%) of myocardial mass downstream of coronary stenosis
and/or
a step of predicting a coronary fractal flow reserve value interval (FFR) by using a second trained deep neural network, applied directly to the detected images or portions of images.

It has a second object a device capable of determining the presence of a coronary stenosis for a patient, comprising:
means for receiving at least one CT curvilinear multiplanar medical image (CT scanner), of said patient including the coronary stenosis;
means for detecting said coronary stenosis on said image or on a portion of said image, by using a first deep neural network;
   characterized in that it further comprises means for predicting a coronary fractal flow reserve value interval by using a second trained deep neural network, applied directly to the detected images or portions of images.

Finally, the invention relates to a non-transitory computer-readable medium storing computer-readable program instructions for determining the presence of coronary stenosis for a patient, comprising execution by a computer-readable program instruction processor having the effect of performing the following operations:
receiving at least one CT curvilinear multiplanar medical image (CT scanner) of said patient including coronary stenosis;
detecting said coronary stenosis on said image or a portion of said image by using a first trained deep neural network;
   characterized in that it further generates by said processor a prediction operation of a coronary fractal flow reserve value interval by using a second trained deep neural network, applied directly to the detected images or portions of images.

In particular, the applicant has been able to develop a method that has qualities for automatically detecting potentially significant coronary stenosis from a hemodynamic viewpoint, based on high-level expertise. In addition, the method has the advantage of being able to predict the hemodynamic character of the stenosis by predicting an FFR threshold with a high probability. This system therefore allows, in a single examination of coronary angiograph, to provide reliable results advantageously allowing to adapt a therapeutic pipe.

The benefit for the patient with respect to existing technologies is therefore very important, since it provides a diagnostic confirmation but especially a prognostic value as well as a suitable therapeutic pipe, without the need for other examinations. Such an information richness allows in particular, in a second time, to simplify the cardiological diagnosis course in an obvious manner Moreover, by reducing the need for other examinations, the potential saving in terms of health expenses is considerable.

In this description, unless otherwise specified, it is understood that, when an interval is given, it includes the upper and lower terminals of said interval.

The invention and the advantages resulting therefrom will be better understood by reading the description and non-limiting embodiments which follow, illustrated in the accompanying drawings in which:

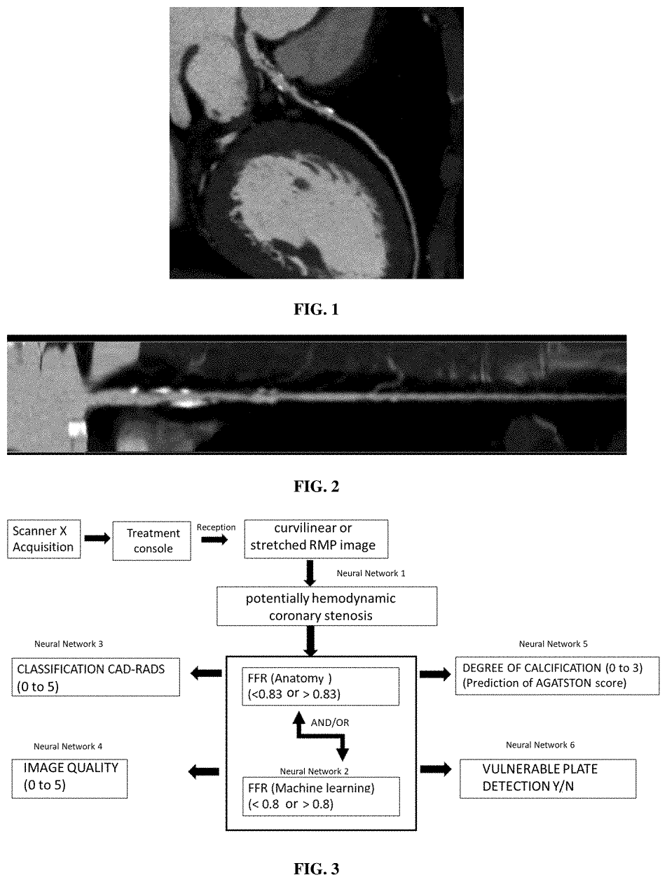

FIG. 1 shows a curvilinear MPR image of a coronary artery having a stenosis.

FIG. 2 shows a stretched MPR image of a coronary artery having a stenosis.

FIG. 3 is a functional diagram illustrating the various possible steps of a method according to the invention.

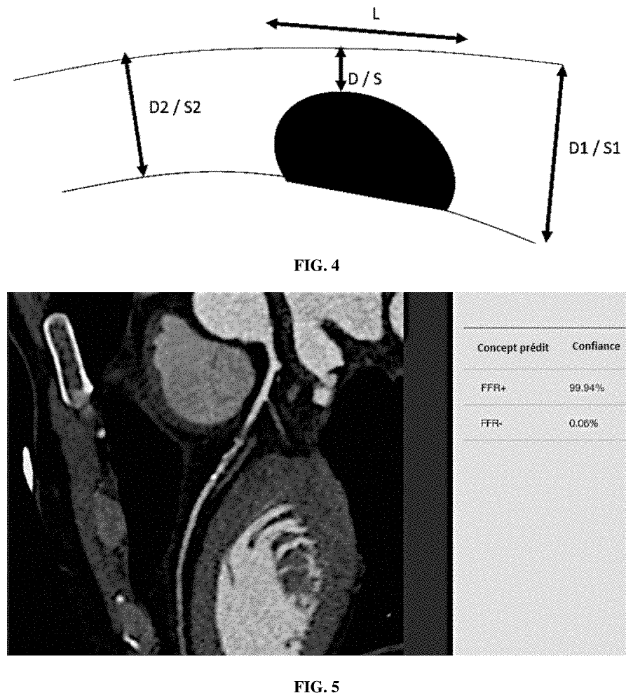

FIG. 4 shows some of the anatomical criteria allowing to predict whether a stenosis is hemodynamic or not.

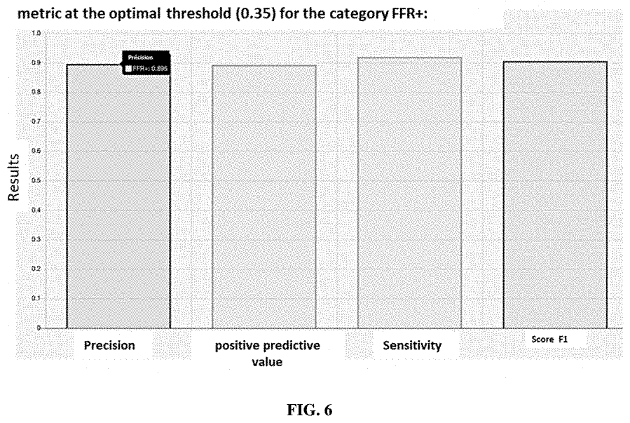

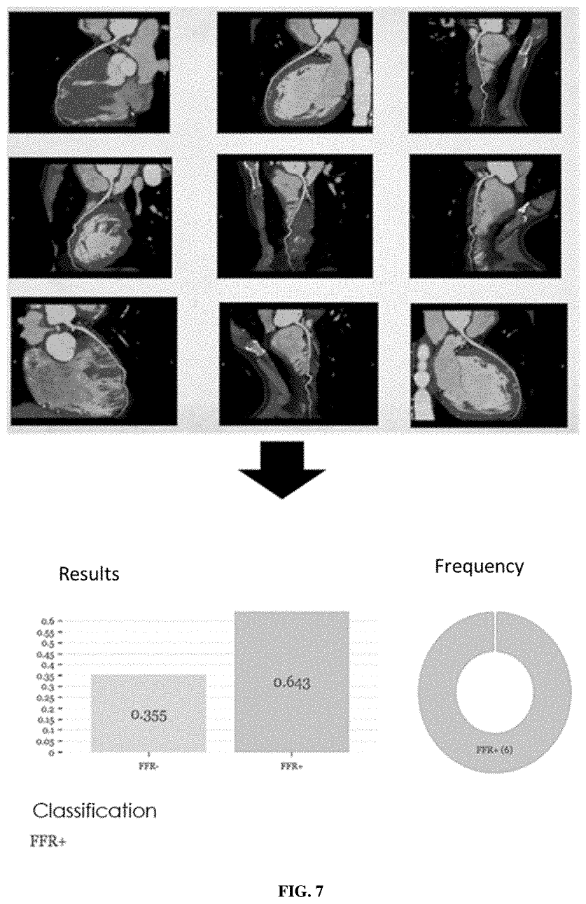

FIG. 5 illustrates the automatic detection of a stenosis with an FFR of less than or equal to 0.8 (FFR+) on a curvilinear MPR image by a second neural network.

FIG. 6 illustrates the performance of the detection of lesions with FFR less than or equal to 0.8 (FFR+) by the second neural network, for one given image.

FIG. 7 illustrates the analysis of multiple images of the same lesion observed under different incidences, enabling a more robust and more efficient classification of the FFR to the threshold of 0.8, compared to the analysis of an isolated image. The techniques of the majority voting or the highest average of the classifications are performant. Nine images of the same artery, seen under different incidences (offset by at least 20°), are analyzed successively. The nine images are classified by the neural network as “FFR+”. The overall final ranking is therefore “FFR+” due to the majority ranking (9/9) and the highest average (0.643) of the “FFR+” ranking.

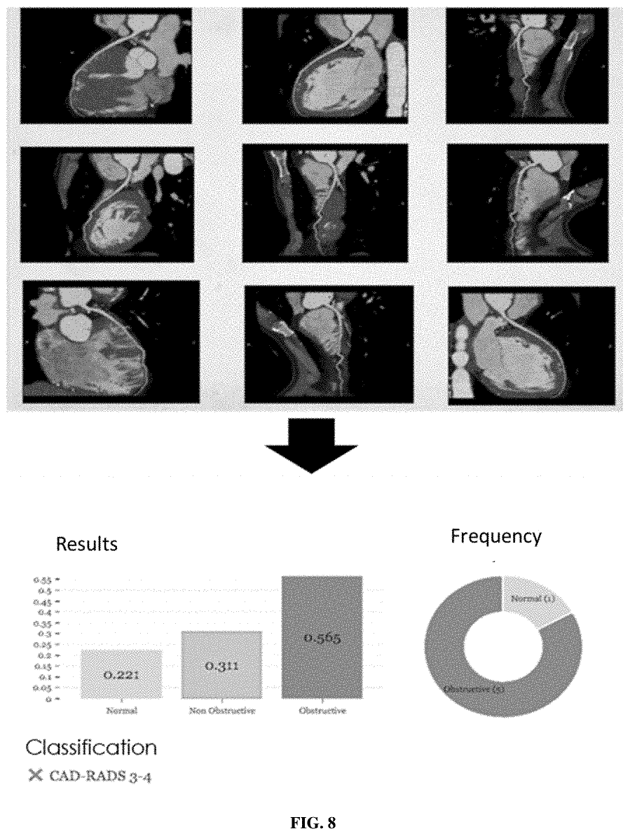

FIG. 8 illustrates the analysis of multiple images of a same lesion observed under different incidences, enabling a more robust and more efficient classification according to CAD-RADS classification, compared to the analysis of an isolated image. The techniques of the majority voting or the highest average of the classifications are efficient. FIG. 8 shows nine images of the same artery, seen under different incidences (shifted by at least 20°), which are analyzed successively. Seven images are classified by the neural network as “normal” (CAD-RADS 0) and two as COPD (CAD-RADS 3 or 4). The overall final ranking is therefore normal (CAD-RADS 0) due to the majority ranking (7/9) and the highest average (0.605) on the nine images.

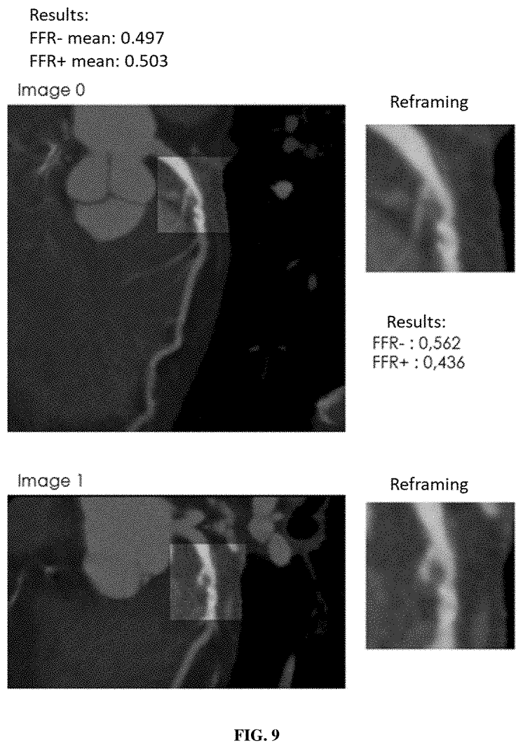

FIG. 9 illustrates the analysis of multiple image fragments of a same lesion observed under different incidences, enabling robust and efficient classification of the FFR to a threshold value of 0.8. The bright zone corresponds to the analyzed image fragment, limited to the coronary lesion, advantageously enabling higher performance of analysis by the neural network. Here the highest average is 0.503 for FFR+. The final classification of the lesion is therefore “FFR+”.

A first object of the invention is a computer-implemented method for determining the presence of coronary stenosis for a patient.

The first step of said method is a step of receiving at least one curved or stretched multiplanar medical image of computed tomography (X-scanner) of said patient including coronary stenosis. A multiplanar image is a reconstructed image from the centerline of a tubular anatomical structure such as a vessel, for example a coronary artery. The major axis of the plane of the image is then aligned with an anatomical structure along this centerline. This allows to include the entire anatomical structure (here a coronary artery) in a single image. A MPR image may follow the curvilinear path of the vessel, the adjacent structures are then distorted. The axis of the vessel can be stretched by projection in a fixed direction. The visualization may be carried out on a 360° rotation axis in the two modes, curvilinear or stretched.

The second step of the method is a step of detecting said coronary stenosis on said image or on a portion of said image by using a first deep neural network.

Preferably, the images or portions of images are derived from a Coronary angiograph (or CCTA for Coronary Computer Tomography).

The first neural network is trained for reading curvilinear MPR images (multiplanar reconstructions), images alone or for more accuracy of the multiple MPR images of the same stenosis according to several incidences, ideally nine incidences of 20° or more of deviation, in order to allow views covering a minimum field of 180°. A basis of at least 5000, preferably 10000 images of coronary arteries has been used, with stenoses classified as potentially hemodynamically significant on the basis of information given by an expert with more than 20 years of experience.

The curvilinear or stretched multiplanar images (multiplanar reconstructions or MPR) are generally used for coronary artery analysis. They are obtained from the central line of a coronary artery. This centerline is extracted by the current software on the radiological workstations, but it is sometimes necessary to correct the central line manually so that this line always remains at the center of the circulating light. Each coronary artery is typically analyzed with multiple MPR by multiplying the incidences over 180 or 360°. The method has been developed and validated with nine images with a minimum of deviation, thus covering 180° to the minimum. This allows in particular to detect the coronary plates with more sensitivity and to quantify the coronary stenoses with more precision.

A known method for automatic detection uses only the stretched MPR images, analyzes the arteries by volume fragments, with a 3 grades classification (normal, less than 50% and greater than 50%), without automated evaluation of the image quality.

For the multiplanar reconstructions (MPR), the raw data is first reconstructed in a plane perpendicular to the z-axis, itself parallel to the major axis of the patient. The resulting image is therefore a cross-section or axial section of a three-dimensional organ. From it, it is possible to select one or more reconstruction planes. The resulting planes may be arbitrary, for example, frontal, coronal and/or sagittal, but they may be oriented according to anatomical or lesional axes. A curvilinear marker conforming to the central line of a vessel such as a coronary artery may allow this structure to be spread along its entire length over a plane reconstruction.

A specific algorithm allows to classify a potentially significant lesion hemodynamically from multiple images. The algorithm, calculates the most frequent classification of the images of a same coronary artery according to different incidences, as well as the average of the probabilities of each classification. In the event of a mismatch classification, the final classification retains the most severe lesion (FFR+) in order to minimize the risk of not treating a patient (false negative risk).

In the averaging calculations, the probability scores of each image less than 0.2 are excluded because they are considered to be low discriminants by the neural network.

Since it is highlighted in particular in FIG. 5, a displayed probability of 0.99 by the neural network indicates that the lesion is very probably with an FFR less than or equal to 0.8, classification confirmed by the actual measured FFR data in this patient (FFR=0.56). Moreover, as it is highlighted in particular in FIG. 6, the optimal decision threshold of 0.35, the overall accuracy of the classification is 89.6%. The neural network thus appears very well to determine the FFR less than the threshold of 0.8.

The method further comprises:
a step of predicting a coronary fractional flow reserve value interval (FFR) by manual, semi-automated and/or automated measurement of at least two morphological criteria chosen from:
   the minimum diameter of the stenosis in mm;
   the minimum surface area of the stenosis in mm²;
   the degree of maximum coronary stenosis expressed as percentage (%) of diameter;
   the degree of maximum coronary stenosis expressed in percentage (%) of surface;
   the length of the stenosis in mm; and/or
   the myocardial mass or the percentage (%) of myocardial mass downstream of the coronary stenosis;
      and/or
a step of predicting a coronary fractal flow reserve value interval by using a second trained deep neural network, applied directly to the detected images or portions of images.

According to a first embodiment of the invention, the method comprises only an additional step of predicting a coronary fractal flow reserve value interval by using a second trained deep neural network, applied directly to the detected images or portions of images.

According to a second embodiment of the invention, the method comprises only an additional step of predicting a coronary fractal flow reserve value interval by manual, semi-automated and/or automated measurement of at least two morphological criteria chosen from:
the minimum diameter of the stenosis in mm;
the minimum surface area of the stenosis in mm²;
the degree of maximum coronary stenosis expressed as percentage (%) of diameter;
the degree of maximum coronary stenosis expressed in percentage (%) of surface;
the length of the stenosis in mm; and/or
the myocardial mass or the percentage (%) of myocardial mass downstream of the coronary stenosis.

According to a third embodiment of the invention, the method comprises both an additional step of predicting a coronary fractal flow reserve value interval by using a second trained deep neural network, applied directly to the detected images or portions of images, and also an additional step of predicting a coronary fractal flow reserve value interval by manual measurement, semi-automated and/or automated of at least two morphological criteria chosen from:
the minimum diameter of the stenosis in mm;
the minimum surface area of the stenosis in mm²;
the degree of maximum coronary stenosis expressed as percentage (%) of diameter;
the degree of maximum coronary stenosis expressed in percentage (%) of surface;
the length of the stenosis in mm; and/or
the myocardial mass or the percentage (%) of myocardial mass downstream of the coronary stenosis.

Preferably, when the lesion is considered potentially significantly hemodynamically, then anatomical criteria are extracted from the image, manually, semi-automatic or automatic:
a: The minimum diameter of the stenosis in mm,
b: the minimum surface area of the stenosis in mm²,
c: the degree of maximum coronary stenosis expressed as percentage (%) of diameter,
d: the degree of maximum coronary stenosis expressed in percentage (%) of surface,
e: the length of the stenosis in mm,
f: the myocardial mass or the percentage (%) of myocardial mass downstream of coronary stenosis.

By manually extracting the image, it must be understood a manual measurement of the diameter and the minimum surface of the vessel (on an artery section image) at the narrowest point of the stenosis; manual drawing or contouring of the diameter and the surface (on an artery section image), at a healthy artery segment the closest to the stenosis upstream and downstream of the stenosis; manually measuring the length of the stenosis, calculating the myocardial mass downstream of a stenosis by segmenting the myocardium downstream of this stenosis; visual estimation of the percentage of myocardium downstream of a stenosis.

By semi-automatic extraction of the image, it must be understood a measure obtained by pre-creating central lines from the pointing of a vessel by the user. At each point of the vessel, the values of the minimum diameter of the surface of the vessel are displayed by an algorithm on the radiological workstation. The various parameters of interest are readable at the area of interest with the possibility of manual correction of the central lines and of the contours. Dedicated specific algorithms can calculate the vascularized myocardium volume downstream of a stenosis (according to the dedicated radiological work console).

By automatic extraction of the image, it must be understood a measurement obtained automatically by automatically creating the lines and contours of the vessel when loading the images of a patient. The measurements are then automatically generated by software. At each point of the vessel, the values of the minimum diameter of the surface of the vessel are displayed by an algorithm on the radiological workstation. The various parameters of interest are readable at the area of interest with the possibility of manual correction of the central lines and contours. Dedicated specific algorithms can calculate the vascularized myocardium volume downstream of a stenosis (according to the dedicated radiological work console).

Advantageously, the combination of at least two of these criteria and the neural network evaluation provides a prediction of the functional character by the FFR above or below the threshold of 0.8.

Preferably, the most relevant anatomical criteria for predicting an FFR value are:
the minimum surface area of the stenosis in mm² and
the degree of maximum coronary stenosis expressed in percentage (%) of surface.

More preferably still, the most relevant anatomical criteria for predicting a value of FFR are:
the minimum surface area of the stenosis in mm²,
the degree of maximum coronary stenosis expressed in percentage (%) of surface, and
the myocardial mass downstream of the stenosis.

Other criteria may also be used to predict an FFR value, it is the following criteria:
the minimum diameter of the stenosis in mm and
the degree of maximum coronary stenosis expressed in percentage (%) of diameter.

As illustrated in FIG. 4, the anatomical criteria for qualifying a stenosis are:
Minimum diameter: D
Minimum area: S
Degree of stenosis in diameter: D/(D1−D2/2)
Surface stenosis degree: S/(S1−S2/2)
Stenosis length: L
Myocardial mass or mass % myocardial mass downstream of a stenosis

The applicant has been able to show, surprisingly, that on a sample of 120 stenoses, at least one of these combinations was in particular capable of separating at 100% of the lesions above or below the threshold value of 0.83 very close to the clinically validated threshold of 0.8.

Advantageously, the step of predicting a coronary fractal flow reserve value interval further comprises using a second trained deep neural network, applied directly to the detected images or portions of images.

The second neural network is trained on reading curvilinear or stretched MPR images, images alone or for more accuracy of the multiple MPR images of the same stenosis according to several incidences. The neural network was successfully trained on coronary images of patients for which the actual value of FFR was measured invasively by intra-coronary pressure sensor.

Analysis of multiple images of coronary stenosis, viewed under different incidences spaced by 20° or more, is advantageous for better artificial intelligence classification of lesions according to the FFR threshold. Indeed, the appearance of a coronary lesion varies according to the incidence of visualization: a lesion can be classified according to an incidence, and FFR-according to another incidence. The classification FFR+ or FFR− of a same image will therefore be different according to each incidence. Compared with the analysis of a single image, the overall analysis of the FFR information on nine incidences increases the robustness and diagnostic accuracy (observed precision gain of approximately 10%). For this, the principle of the majority vote of each FFR classification is used, and/or the average of the classification scores of each image (FIG. 7). In order to improve the robustness, it is excluded from the majority vote or on the average certain images that have not reached a minimum probability threshold FFR for each category (for example a probability <0.2).

The analysis by combining anatomical criteria is added to the artificial Intelligence analysis. If the two analyses classify the lesion in the same way, it is this ranking that is proposed to the user.

In the event of a mismatch between anatomical criteria and artificial intelligence criteria, this is the FFR+ ranking that is proposed to decrease the risk of false negatives, since it is considered to be more severe not to diagnose a treatable lesion and to raise a lesion (thus promoting the sensitivity of the diagnosis of hemodynamic stenoses).

Thus, for example:

a/ Nine MPR images of a same artery with stenosis identified by the first neural network are input to the algorithm. The latter finds five images FFR classified “+” and four images FFR classified “−”. The average probability FFR “+” is 0.6, that FFR “−” is 0.5. In this case, the lesion is classified as FFR “+” (hemodynamically significant) because the FFR “+” ranking is more frequent and the average probability FFR “+” is higher. The lesion will be classified according to a probability of 0.6.

b/ Seven other MPR images of a second artery with stenosis identified by the first neural network are input to the algorithm. Six are FFR “−”, with an average probability of 0.9, the last one is FFR “+” with an average probability of 0.7. The ranking is more frequently FFR “−” with the highest probability. The stenosis is then judged to be hemodynamically significant (with a high probability: 0.9).

In the averaging calculations, the probability scores less than 0.2 are excluded because they are considered to be low discriminants by the neural network.

The result of this second neural network, if it confirms the first prediction on anatomical criteria, makes the prediction highly probable. In the event of mismatch, the result of the FFR+ prediction overrides in order to favor the detection sensitivity by minimizing the number of false negatives. It is indeed considered to be more serious to sub-estimate a lesion that overestimate.

The method according to the invention may advantageously also comprise a step of determining a value according to the CAD-RADS classification (for Coronary Artery Disease-Reporting and Data System value or System of reports and Data) of a Coronary stenosis by using a third trained deep neural network, applied directly to the detected images or portions of images.

Said third neural network was successfully trained by supervised learning on arteries in which CAD-RADS classification was applied by a recognized expert.

A specific algorithm allows to classify a coronary artery according to CAD-RADS from multiple images. The algorithm, takes the most frequent classification of the images of different incidences (from 0 to 5), and compares it to the average of the probabilities of each classification. If the most frequent classification is also that with the highest average probability, it is retained by the algorithm. In the event of mismatch, it is the most frequent ranking that is retained. The algorithm eliminates calculation of the probability scores below a certain decision threshold, previously defined in order to optimize the diagnostic performance of the neural network. Analysis of multiple images of coronary stenosis, viewed under different incidences spaced by 20° or more, is advantageous for better classification of lesions according to their CAD-RADS classification. Indeed, coronary lesions (plates or stenoses) are often not developed symmetrically. Consequently, the appearance of the lesion varies according to the proposed incidence: a lesion could appear tight according to an incidence, less tight or even sometimes not existing according to a third incidence. The ranking of a same image will therefore be different according to its incidence. Compared with the analysis of a single image, the overall analysis of CAD-RADS information on nine incidences increases the robustness and accuracy (observed gain of 10% accuracy). For this, the principle of the majority vote of each CAD-RADS classification per image is used, and/or the average of the classifications of each image. In order to improve the robustness, it is excluded from the vote or on the average certain images that have not reached a minimum probability threshold for each CAD-RADS category.

Thus, for example:

a/ nine MPR images of a same artery are classified by the third neural network. Five MPR images are classified as CAD-RADS 4, two MPR images are classified as CAD-RADS 3, two MPR images are classified as CAD-RADS 2. The average probability CAD-RADS 4 is 0.8, that CAD-RADS 3 is 0.5, that of CAD-RADS 2 is 0.3. In this case, the lesion is classified CAD-RADS 4 because this ranking is more frequent and its mean probability CAD-RADS 4 is higher. The lesion will be classified as CAD-RADS 4 with a probability of 0.8 (strong confidence).

b/ Seven other MPR images of a same artery are classified by the third neural network. Four MPR images are classified as CAD-RADS 2, three MPR images are classified as CAD-RADS 3. The average probability CAD-RADS 2 is 0.6, that CAD-RADS 3 is 0.7. In this case, the lesion is classified CAD-RADS 2 because this ranking is more frequent. The lesion will be classified as CAD-RADS 2 with a probability of 0.6 (with low confidence).

In the averaging calculations, the probability scores less than 0.2 for a category are excluded because they are considered to be too little discriminant by the neural network.

The CAD-RADS classification enables rational and uniformized classification of atheromatous coronary lesions. This 6-degree classification of severity (from 0 to 5) allows to propose optimal therapeutic choices for the patient to the light of the results of the coronary scanner.

At this day, no automatic classification based on CAD-RADS was proposed. Such automatic detection, if it is reliable, is particularly very useful for facilitating the daily work of interpretation of radiologists or cardiologists.

Advantageously, the CAD-RADS classification reinforces the predicted value of the FFR, because the CAD-RADS classifications of less than 3 correspond to the stenoses less than 50%.

Therefore, a CAD-RADS score less than 3 is probably not associated with an FFR less than or equal to 0.8.

Conversely, A CAD-RADS classification 4 greatly enhances the probability of an FFR value less than or equal to 0.8.

The method according to the invention thus advantageously comprises a CAD-RADS classification for each curvilinear MPR image of a same artery at different angles.

For a group of one to nine images, the average probability score of each CAD-RADS category is calculated as well as the most frequent CAD-RADS category. If the category that obtains the best score is also the most frequent category, it is retained as the most probable classification (FIG. 8). Otherwise, the most severe result of the two rankings is retained, in order to minimize the risk of underestimate a lesion. The overestimation is considered to be more dangerous than the summit for the detection of coronary lesions.
CAD-RADS 0: normal
CAD-RADS 1: plaque <25%
CAD-RADS 2: plaque between 25 and 49%
CAD-RADS 3: 50-69% (stenosis)
CAD-RADS 4: 70-99% (stenosis)
CAD-RADS 5: occlusion

Preferably, for a more robust classification the categories 1 and 2 and the categories 3 and 4 can be grouped as follows, according to the desire of the end user.
CAD-RADS 0: normal
CAD-RADS 1 or 2: non-obstructive coronary artery disease
CAD-RADS 3 or 4: obstructive coronary artery disease
CAD-RADS 5: occlusion

The method according to the invention advantageously comprises at least one of the following steps, which can be performed in any order:
a step of automated determination of the image quality providing a diagnostic confidence index by using a fourth trained neural network, applied directly to the detected images or portions of images;
a step of determining a global calcification score on a scale of 0 to 4 predicting the category of the Agatston calcium score, by using a fifth trained neural network, applied directly to the detected images or portions of images; and/or
a step of determining a high-risk plaque (HRP) of a cardiac event, by using a sixth network of trained neurons, applied directly to the detected images or portions of images.

An excellent image quality is advantageous in order to obtain a reliable, relevant and accurate diagnosis in CCTA. The presence of artifacts, related to cardiac motion, to insufficient contrast, or to noise in the image (the noise is measured as the standard deviation of the pixel values in a homogeneous region of an image), interfere with the diagnosis and quantification of coronary stenosis and make the therapeutic decisions that follow more difficult. An automatic image quality assessment is useful for quality control, and for comparing the images from one center to the other. In the present invention, this automatic evaluation is advantageously used to provide a diagnostic confidence index in the final interpretation.

The fourth neural network for the step of automated image quality determination was successfully trained by supervised learning on images arteries whose image qualities were evaluated by a recognized expert. The images were classified according to a score of 0 to 4 according to the subjective scale (detailed below).

The neural network provides an overall image quality score from one to nine images of the same artery. The average value of the image quality scores is calculated. This value defines a confidence index between 0 and 4.

A specific algorithm allows to classify the image quality from multiple images of the same artery. The algorithm, takes the average of the classifications of the images of an artery according to different incidences (classified from 0 to 4).

Thus, for example:

a/ nine MPR images of a same artery are classified by the fourth neural network. Five MPR images are classified IQ 4, four MPR images are classified IQ 3.

The retained quality will be (5*4+4*3)/9=3.6. This number is, for example, considered a confidence indicator for the final analysis.

The classification of the image quality is resumed below:
IQ=0-Non-evaluable
IQ=1-Low image quality. Artifact presence. Low diagnostic confidence
IQ=2. Correct. Interpretation is possible but the degree of confidence is low
IQ=3. Good IQ. Good diagnostic confidence
IQ=4. Excellent IQ. High degree of confidence

The fifth network for the step of determining a global calcification score was trained directly on MPR images of arteries for which the Agatston calcium score was known by a prior scanner examination without contrast product injection. After training, the degree of calcification is predicted semi-quantitatively on an injected angiograph, according to four categories, for each of the extracted arteries:
0: Calcification step
1: Moderate Calcifications: Agatston calcium score predicted between 1 and 99
2: Average Calcifications: Agatston calcium score predicted: between 100 and 400
3: Severe Calcifications: Agatston calcium score predicted: greater than 400

The detection of coronary calcium is documented in the literature: in particular the Agatston score is recognized as a large and independent risk marker for predicting coronary events, as well as risk factors known as the high level of cholesterol, diabetes or hypertension.

An algorithm retains the highest score on the images of a same artery according to multiple incidences, and then makes the sum of the scores obtained for each artery to obtain a global calcification score, allowing to predict a risk:
Sum=0 predicted Agatston calcium score: zero
Sum=1 predicted Agatston calcium score: 1-100
Sum=2 predicted Agatston calcium score: 100-200
Sum=3 predicted Agatston calcium score: 200-400
Sum>=4 predicted Agatston calcium score: >400

In the literature, the Agatston calcium score allows to estimate the risk of cardiovascular event at 10 years:
0: minimal risk,
Less than 100: low risk,
100-400: intermediate risk,
400: high risk.

According to the method of the invention, the calcium score is calculated beforehand on a scanner without contrast injection, because the high-density contrast interferes with the detection of calcium. Machine learning was used to estimate the score automatically on contrast examinations, by previously provided the score obtained on a scanner without contrast of the same patient.

Thus, for example:

For a given patient, five images are analyzed for each main artery (IVA for the anterior interventricular artery, Cx for the circumflex artery and RC for the right coronary)

The calcium scores are as follows:
IVA: 0, 0, 0, 0, 1
Cx: 1, 1, 1, 2, 1
RC: 0, 0, 0, 0, 0

Thus, the score

$$\begin{matrix}Ca = Max ( IVA ) + Max ( Cx ) + Max ( RC ) \\ = 1 + 2 + 0 \\ = 3\end{matrix}$$

The score being equal to 3, the predicted Agatston calcium score will be between 200 and 400, corresponding to an intermediate risk.

Finally, the sixth network for the high-risk plaque determination step was successfully trained by supervised learning on MPR images or section images of arteries perpendicular to the MPR images (cross sectional images) in which the possible presence of a vulnerable plate was detected by a recognized expert. After training, the presence of a vulnerable plate is asserted according to a probability threshold (between 0 and 1) defined as the optimal threshold for the performance of this neural network.

The risk plaques (HRP) are characterized by the presence of the following elements: low density plate (LDP), positive reshaping plate (PR) by increasing the wall of the vessel to the outside, presence of a negative density zone within the plate (lipid core).

The plate has most often an asymmetric character.

The neural network uses MPR images which may be optionally supplemented by artery section images at the plate (such as CROSS SECTIONAL IMAGES), in order to comfortably sense the accuracy of the detection.

There is no system based on deep learning which automatically detects the risk plaques from MPR images.

The presence of a vulnerable plate is retained if the score is greater than the threshold thus defined on at least one of the MPR images or on an artery section image.

A specific algorithm allows to determine the presence of a vulnerable plate. Due to its asymmetric character, it may be that a plate is not visible on one or more incidences of MPR images due to a different angle of view.

Thus, for example:

a/ Five MPR images of a same artery are analyzed by the sixth neural network. The presence of a vulnerable plate is denoted V, if the probability threshold reaches or exceeds 0.5, its absence is denoted by 0.

The result 0, 0, 0, V, 0 corresponds to the presence of a vulnerable plate (because the presence has been detected on at least one image MPR).

Preferably, as illustrated in FIG. 3, the method of the invention comprises the following five steps which can be performed in any order:
a step of predicting a coronary fractal flow reserve interval by using a second trained deep neural network, applied directly to the detected images or portions;
a step of determining a value according to the CAD-RADS classification by using a third trained deep neural network, applied directly to the detected images or portions of images;
a step of automated determination of the image quality providing a diagnostic confidence index from a fourth trained neural network, applied directly to the detected images or portions of images;
a step of determining a global calcification score on a scale of 0 to 4 predicting the Agatston calcium score, using a fifth trained neural network, applied directly to the detected images or portions of images; and
a step of determining a high-risk plaque of a cardiac event, using a sixth trained neural network, applied directly to the detected images or portions of images.

The invention also relates to a device capable of determining the presence of a coronary stenosis for a patient, comprising:
means for receiving at least one CT curvilinear multiplanar medical image (CT scanner), of said patient including the coronary stenosis;
means for detecting said coronary stenosis on said image or on a portion of said image, by using a first deep neural network.

Said device further comprises means for predicting a coronary fractal flow reserve interval by using a second trained deep neural network applied directly to the detected images or portions of images.

The first and second neural networks are as described above.

Preferably, the device further comprises means for determining a value according to the CAD-RADS classification by using a third trained deep neural network, applied directly to the detected images or portions of images.

The third neural network is as described above.

Advantageously, the device according to the invention further comprises at least one of the following means:
means for automated determination of the image quality providing a diagnostic confidence index by using a fourth trained neural network, applied directly to the detected images or portions of images;
means for determining a global calcification score on a scale of 0 to 4 predicting the Agatston calcium score, by using a fifth neural network trained, applied directly to the detected images or portions of images; and/or
means for determining a high-risk plaque of a cardiac event, by using a sixth network of trained neurons, applied directly to the detected images or portions of images.

The fourth, fifth and sixth neural networks are as described above.

According to a preferred embodiment of the invention, the device comprises the following five means:
means for predicting a coronary fractal flow reserve interval by using a second trained deep neural network, applied directly to the detected images or portions;
means for determining a value according to the CAD-RADS classification by using a third trained deep neural network, applied directly to the detected images or portions of images;
means for automated determination of the image quality providing a diagnostic confidence index by using a fourth trained neural network, applied directly to the detected images or portions of images;
means for determining a global calcification score on a scale of 0 to 4 predicting the Agatston calcium score, by using a fifth neural network trained, applied directly to the detected images or portions of images; and
means for determining a high-risk plaque of a cardiac event, by using a sixth network of trained neurons, applied directly to the detected images or portions of images.

Finally, the invention relates to a non-transitory computer-readable medium storing computer-readable program instructions for determining the presence of coronary stenosis for a patient, comprising the execution by a computer-readable program instruction processor having the effect of performing the following operations:
receiving at least one CT curvilinear multiplanar medical image (CT scanner) of said patient including coronary stenosis;
detecting said potentially hemodynamically significant coronary stenosis on said image or a portion of said image by using a first trained deep neural network;
   characterized in that it further generates by said processor a prediction operation of a coronary fractal flow reserve value interval by using a second trained deep neural network, applied directly to the detected images or portions of images.

Preferably, the support is able to further generate, by said processor, an operation of determining a value according to the CAD-RADS by using a third trained deep neural network, applied directly to the detected images or portions of images.

More preferably, the support is able to further generate, by said processor, at least one of the following operations:
automated determination of the image quality providing a diagnostic confidence index by using a fourth trained neural network, applied directly to the detected images or portions of images;
determining a global calcification score on a scale of 0 to 4 predicting the Agatston calcium score, by using a fifth trained neural network, applied directly to the detected images or portions of images; and/or
determining high risk plaque, using a sixth trained neural network, applied directly to the detected images or portions of images.

According to a preferred embodiment of the invention, the support generates the embodiment by said processor of the following five operations:
predicting a coronary fractal flow reserve value interval by using a second trained deep neural network, applied directly to the detected images or portions;
determining a value according to the CAD-RADS classification by using a third trained deep neural network, applied directly to the detected images or portions;
automated determination of the image quality providing a diagnostic confidence index by using a fourth trained neural network, applied directly to the detected images or portions of images; determining a global calcification score on a scale of 0 to 4 predicting the Agatston calcium score, by using a fifth trained neural network, applied directly to the detected images or portions of images; and
determining high risk plaque, using a sixth trained neural network, applied directly to the detected images or portions of images.

The present invention will now be illustrated by the following examples:

### Example 1

A wide database of more than at least 5000, preferably more than 10,000 MPR images from coronary angiograph has been used for supervised learning. All images were classified and labeled by an expert with more than 20 years of experience of reading these images (cumulative experience of about 50,000 cases analyzed). The images of this base were classified in terms of image quality, degree of calcification, and degree of stenosis as a function of CAD-RADS classification. The possible presence of risk plaque (vulnerable plate) has been specified. Moreover, over more than 2000, preferably 4000 images of patients with stenosis and known FIR value, a binary classification at the threshold FFR of 0.8 was performed. A neural network could thus be trained (over 80% of the images) to predict on a new image (among the 20% of the remaining images, serving as a test basis) if the value of FFR will be higher or lower (> or <) to 0.8.

Different networks of available neurons in free access have been tested: GOODGLENET™, RESNET™ and INCEPTION™ V3, VGG11™, VGG13™, VGG19™ to obtain the best classification rate. Various measurements were made on a test database, independent of the learning base: precision calculations of the test, sensitivity, specificity, positive predictive value, F1 score (harmonic mean between sensitivity and positive predictive value), area under the ROC Curve (Receive Operator Curve, with sensitivity in abscissa and 1-specificity in ordinate).

An area under the curve reaching 0.85 was obtained, resulting in a good overall predictive performance, equal to or greater than the current predictive systems. The scores should progress with the progressive increase of the drive base.

The MPR multiplanar images are provided from the base by the radiological post-processing consoles, from the central lines. Curvilinear or stretched MPR images, one to nine images of the same artery. The images may be exported from the workstations in a standard image format (ex: JPEG, or PNG), and secondarily loaded on a dedicated Internet site. They may also be directly loaded on an Internet site from the workstation to the DICOM standard X-ray format. The evaluation result produced is then returned to the usual environment of the reader. The neural networks may also be directly integrated with the post-processing consoles.

### Example 2: Creation of Neural Networks, Methods and Results

A wide database of stretched or curvilinear MPR images was created from examinations made on different scanners.

The patient data has been anonymized.

For each dataset, the MPR images of the three main arteries have been extracted, namely: IVA, the circumflex artery, and the right coronary artery.

For each of the three arteries, nine images were selected with different angles of view (20° of minimum deviation between two images), by rotation around the centerline.

Each MPR image was classified by an expert in view of the image quality, the degree of calcification, the presence or absence of a vulnerable plate, and its degree of stenosis using the CAD-RADS classification.

A reduced database of more than 1800, preferably 4500 images over more than 200, preferably 500 patients with a known FFR value was also driven.

The images were loaded on a platform (DEEPOMTIC™ STUDIO, Paris, France and CLEVERDOC, Lille, France) for classifying the images and testing the different neural networks.

For each neural network, 80% of the images of the database were used for training, and 20% of the images were used for evaluation. The evaluation images have been excluded from the training process.

For each task, different neural networks have been used for training.

The networks associated with the best results (better sensitivity and better positive predictive value) have been selected.

### RESULTS AND CONCLUSION

For the automatic detection of the vulnerable plates, the average score F1 has reached 60%.

For the automatic calcium score, the average score F1 reaches 75%.

For the automatic classification of the image quality, the score F1 reaches 75%.

For the classification of the stenoses according to CAD-RADS, the average score F1 reaches 87%.

For the prediction of the FFR at the threshold of 0.8, the average F1 score reaches about 85% or even 87%.

The results obtained on this first base therefore appear equal to or greater than those of the other methods which are more complex to implement and more expensive.

1.-13. (canceled)

14. A computer-implemented method for determining the presence of coronary stenosis for a patient, comprising:
receiving at least one curvilinear or stretched multiplanar medical image of computed tomography (X-scanner) of said patient including coronary stenosis;
detecting said coronary stenosis on said image or on a portion of said image by using a first trained deep neural network;
predicting a coronary fractal flow reserve interval (FFR) by manual, semi-automated and/or automated measurement of at least two morphological criteria chosen from:
the minimum diameter of the stenosis in mm;
the minimum surface of the stenosis in mm²;
the degree of maximum coronary stenosis expressed in percentage (%) of diameter;
the degree of maximum coronary stenosis expressed in percentage (%) of surface;
the length of the stenosis in mm; and/or
the myocardial mass or the percentage (%) of myocardial mass downstream of the coronary stenosis; and/or
predicting a coronary fractal reserve interval (FFR) by using a second trained deep neural network, applied directly to the detected images or portions of detected images.

15. The method according to claim 14, further comprising determining a value according to a CAD-RADS classification (for Coronary Artery Disease-Reporting and Data System value or System of reports and Data) of coronary stenosis by using a third trained deep neural network, applied directly to the detected images or portions of detected images.

16. The method according to claim 14, further comprising at least one of the following:
automated determination of the image quality providing a diagnostic confidence index by using a fourth trained neural network, applied directly to the detected images or portions of detected images;
determining a global calcification score on a scale of 0 to 4 predicting the Agatston calcium score, by using a fifth trained neural network, applied directly to the detected images or portions of detected images; and/or
determination a high-risk plaque (HRP) of cardiac event, by using a sixth trained neural network, applied directly to the detected images or portions of detected images.

17. The method according to claim 16, further comprising:
predicting a coronary reserve flow value interval by using a second trained deep neural network, applied directly to the detected images or portions;
determining a value according to the CAD-RADS classification by using a third trained deep neural network, applied directly to the detected images or portions of detected images;
automated determination of the image quality providing a diagnostic confidence index from a fourth trained neural network, applied directly to the detected images or portions of images; a step of determining a global calcification score on a scale of 0 to 4 predicting the Agatston calcium score, using a fifth trained neural network, applied directly to the detected images or portions of detected images; and
determining a high-risk plaque of a cardiac event, using a sixth neural network trained, applied directly to the detected images or portions of detected images.

18. The method according to claim 14, wherein the images or portions of images are derived from a coronary angiography (or CCTA for Coronary Computer Tomography).

19. A device for determining the presence of coronary stenosis for a patient, comprising:
at least one input adapted to receive at least one CT curvilinear multiplanar medical image (X scanner), of said patient including coronary stenosis; and
at least one processor configured for:
detecting said coronary stenosis on said image or a portion of said image, by using a first trained deep neural network; and predicting a coronary fractal flow reserve interval by using a second trained deep neural network, applied directly to the detected images or portions of images.

20. The device according to claim 19, wherein the at least one processor is further configured for determining a value according to the CAD-RADS classification by using a third trained deep neural network, applied directly to the detected images or portions of images.

21. The device according to claim 19, wherein the at least one processor is further configured for:
automated determination of image quality providing a diagnostic confidence index by using a fourth trained neural network, applied directly to the detected images or portions of images;
determining a global calcification score on a scale of 0 to 4 predicting the Agatston calcium score, by using a fifth trained neural network, applied directly to the detected images or portions of images; and/or
determining a high-risk plaque of a cardiac event, by using a sixth network of trained neurons, applied directly to the detected images or portions of images.

22. The device according to claim 21, wherein the at least one processor is further configured for:
predicting a coronary fractal flow reserve interval by using a second trained deep neural network, applied directly to the detected images or portions;
determining a value according to the CAD-RADS classification by using a third trained deep neural network, applied directly to the detected images or portions;
automated determination of image quality providing a diagnostic confidence index by using a fourth trained neural network, applied directly to the detected images or portions of images;
determining a global calcification score on a scale of 0 to 4 predicting the Agatston calcium score, by using a fifth trained neural network, applied directly to the detected images or portions of images; and
determining a high-risk plaque of a cardiac event, by using a sixth network of trained neurons, applied directly to the detected images or portions of detected images.

23. A non-transitory computer-readable medium storing computer-readable program instructions for determining the presence of coronary stenosis for a patient, comprising executing by a computer-readable program instruction processor having the effect of performing the following operations:
receiving at least one CT curvilinear multiplanar medical image (X-scanner) of said patient including coronary stenosis;
detecting said potentially hemodynamically significant coronary stenosis on said image or a portion of said image by using a first trained deep neural network;
wherein the non-transitory computer-readable medium further generates by said processor a prediction operation of a coronary reserve flow value interval by using a second trained deep neural network, applied directly to the detected images or portions of detected images.

24. The non-transitory computer-readable medium according to claim 23, wherein the non-transitory computer-readable medium further generates by said processor an operation of determining a value according to the CAD-RADS classification by using a third trained deep neural network, applied directly to the detected images or portions of detected images.

25. The non-transitory computer-readable medium according to claim 23, wherein the non-transitory computer-readable medium further generates, by said processor, at least one of the following operations:
automated determination of image quality providing a diagnostic confidence index by using a fourth trained neural network, applied directly to the detected images or portions of detected images;
determination of a global calcification score on a scale of 0 to 4 predicting the Agatston calcium score, by using a fifth trained neural network, applied directly to the detected images or portions of detected images; and/or
determination of a high-risk plaque of a cardiac event, by using a sixth network of trained neurons, applied directly to the detected images or portions of detected images.

26. The non-transitory computer-readable medium according to claim 25, wherein the non-transitory computer-readable medium generates the execution by said processor of the following five operations:
predicting a coronary fractal flow reserve interval by using a second trained deep neural network, applied directly to the detected images or portions of detected images;
determining a value according to the CAD-RADS classification by using a third trained deep neural network, applied directly to the detected images or portions of detected images;
determining automatically an image quality providing a diagnostic confidence index by using a fourth trained neural network, applied directly to the detected images or portions of detected images;
determining a global calcification score on a scale of 0 to 4 predicting the Agatston calcium score, by using a fifth trained neural network, applied directly to the detected images or portions of detected images; and
determining a high-risk plaque of a cardiac event, by using a sixth network of trained neurons, applied directly to the detected images or portions of detected images.
